(19) United States
(12) Patent Application Publication
(10) Pub. No.: US 2024/0327768 A1
(43) Pub. Date: Oct. 3, 2024

(54) MICROFLUIDIC DEVICE MIMICKING TUMOR MICROENVIRONMENT

(71) Applicant: The Texas A&M University System, College Station, TX (US)

(72) Inventors: Abhishek Jain, Cypress, TX (US); James J. Tronolone, Bryan, TX (US); Biswajit Saha, Bryan, TX (US)

(73) Assignee: The Texas A&M University System, College Station, TX (US)

(21) Appl. No.: 18/574,081

(22) PCT Filed: Jul. 7, 2022
(86) PCT No.: PCT/US22/36395

§ 371 Date: Dec. 26, 2023

Related U.S. Application Data
(60) Provisional application No. 63/219,827, filed on Jul. 8, 2021.

(51) Int. Cl. C12M 3/06; C12M 1/12; C12M 3/00

(52) U.S. Cl. CPC C12M 23/16; C12M 21/08; C12M 25/14

(57) ABSTRACT

In an embodiment, the present disclosure pertains to a medical device. In some embodiments, the medical device includes a first compartmentalized microfluidic channel flanked by two microchannels and a second compartmentalized microfluidic channel. In some embodiments, the first and second compartmentalized microfluidic channels are situated vertically one to another. In some embodiments, the medical device further includes a porous membrane situated between the first and second compartmentalized microfluidic channels. In another embodiment, the present disclosure pertains to an organ-on-chip. In some embodiments, the organ-on-chip includes a top portion having three parallel microchannels. In some embodiments, the three parallel microchannels are separated by micropillars. In some embodiments, the organ-on-chip further includes a bottom portion having a fourth microchannel and a membrane between the top portion and the bottom portion.

### CROSS-REFERENCE TO RELATED APPLICATIONS

This patent application claims priority from, and incorporates by reference the entire disclosure of, U.S. Provisional Application No. 63/219,827 filed on Jul. 8, 2021.

### TECHNICAL FIELD

The present disclosure relates generally to microfluidic devices and more particularly, but not by way of limitation, to microfluidic devices mimicking tumor microenvironments.

### BACKGROUND

This section provides background information to facilitate a better understanding of the various aspects of the disclosure. It should be understood that the statements in this section of this document are to be read in this light, and not as admissions of prior art.

Platelets extravasate from the circulation into tumor microenvironments, enable metastasis and confer resistance to chemotherapy in several cancers. Therefore, arresting tumor-platelet crosstalk with effective and atoxic antiplatelet agents in combination with anticancer drugs may serve as an effective cancer treatment strategy. To test this concept, a new ovarian tumor microenvironment-chip (OTME-Chip) was created that is composed of a platelet-perfused tumor microenvironment and which recapitulates platelet extravasation and its consequences. By including gene-edited tumors and next generation RNA-seq on-chip, the OTME-Chip revealed that platelets and tumors interact through glycoprotein GPVI and tumor galectin-3 under shear in time-dependent manner. Finally, as proof-of-principle of a clinical trial, it is shown that a GPVI inhibitor, Revacept, impairs metastatic potential and improves chemotherapy. Since GPVI is an antithrombotic target which does not impair hemostasis, it represents a safe cancer therapeutic. As such, it is proposed that TME-Chip could be deployed to study other vascular and hematological targets in cancer.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a medical device. In some embodiments, the medical device includes a first compartmentalized microfluidic channel flanked by two microchannels and a second compartmentalized microfluidic channel. In some embodiments, the first and second compartmentalized microfluidic channels are situated vertically. In some embodiments, the medical device further includes a porous membrane situated between the first and second compartmentalized microfluidic channels.

In some embodiments, the first and second compartmentalized microfluidic channels are in fluid communication such that cells can migrate from the first compartmentalized microfluidic channel to the second compartmentalized microfluidic channel through the porous membrane. In some embodiments, the first compartmentalized microfluidic channel includes cancer cells. In some embodiments, the second compartmentalized microfluidic channel includes blood vessel-mimicking endothelium. In some embodiments, the two microchannels include a biologically derived hydrogel scaffold. In some embodiments, the biologically derived hydrogel scaffold permits for at least one of time-lapse visualization of perfused platelets within a vessel, their extravasation through an endothelium, or consequential cancer cell invasiveness. In some embodiments, the medical device further includes lateral channels separated by a plurality of micropillars. In some embodiments, the plurality of micropillars are at a set distance to facilitate formation of a solid-liquid interface. In some embodiments, the solid-liquid interface facilitates in two-dimensional monolayers of tumor cells assuming a three-dimensional morphology. In some embodiments, the three-dimensional morphology is assumed after undergoing epithelial-to-mesenchmyal transition (EMT). In some embodiments, the medical device identifies an antiplatelet therapeutic. In some embodiments, the antiplatelet therapeutic is Revacept.

In another embodiment, the present disclosure pertains to an organ-on-chip. In some embodiments, the organ-on-chip includes a top portion having three parallel microchannels. In some embodiments, the three parallel microchannels are separated by micropillars. In some embodiments, the organ-on-chip further includes a bottom portion having a fourth microchannel and a membrane between the top portion and the bottom portion.

In some embodiments, the micropillars include hexagonal micropillars. In some embodiments, each micropillar has dimensions of 250 μm×100 μm×100 μm (length×width×height). In some embodiments, each micropillar are equally spaced apart leaving 100 μm between each micropillar. In some embodiments, the top portion include cancer cells. In some embodiments, the bottom portion includes blood vessel-mimicking endothelium. In some embodiments, the micropillars include a biologically derived hydrogel scaffold. In some embodiments, wherein the biologically derived hydrogel scaffold permits for at least one of time-lapse visualization of perfused platelets within a vessel, their extravasation through an endothelium, or consequential cancer cell invasiveness. In some embodiments, the organ-on-chip identifies an antiplatelet therapeutic. In some embodiments, the antiplatelet therapeutic comprises Revacept.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIGS. 1A-1D illustrate a microdevice containing two polydimethylsiloxane (PDMS) compartments separated by a thin porous membrane that reproduces the microarchitecture of the tumor-vascular interface. FIG. 1A is a perspective view of the microdevice, FIG. 1B is a top view of the microdevice, FIG. 1C is an exploded assembly of the microdevice, and FIG. 1D is a detail view of the two PDMS compartments.

FIGS. 2A and 2B are sectioned side views of a microdevice illustrating platelet triggered OvCa cell migration.

FIG. 3 is a timeline illustrating steps of TME-Chip formation, platelet extravasation into the tumors, and following consequences. Cancer cell dynamics and molecular readouts are analyzed every 24 hr post-platelet extravasation.

FIG. 4 is a schematic diagram of TME-Chip showing tumor invasion dynamics can be systematically visualized and characterized after platelet extravasation from the bottom vascular chamber into the top tumor chamber.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Platelets have long been recognized as anuclear blood constituents that regulate hemostasis and thrombosis. Interestingly, platelets are also the first responders in the pathobiology of cancer: they play both structural and functional roles as reporters and transporters within the tumor-vascular organ. To date, in vivo systems have been widely used in the understanding of cancer metastasis and its control through platelet function. For example, both mouse models as well as human clinical studies of ovarian cancer have shown that platelets extravasate into the solid tumor microenvironment and facilitate proliferation. A few animal studies have proposed the link between platelet extravasation and metastasis in ovarian cancer. Some reports also indicate that platelet surface receptor GPVI interacts with circulating tumor cell (CTC) surface glycoprotein galectin-3, and potentiates its growth. But a more detailed understanding of pathophysiological outcomes of trans-endothelial platelet extravasation in cancer remains limited. Importantly, whether extravasated platelets can affect primary solid tumors through their GPVI remains questionable. Additionally, because vascular shear regulates platelet GPVI activation, it remains to be tested if shear can also regulate platelet interaction with cancer cells. Extravasated platelets are also known to release tumor-promoting growth factors and cytokines/interleukins, and induce chemoresistance in in vivo models. Therefore, antiplatelet therapeutics in cancer offered along with chemotherapy can be helpful. But while the in vivo models have provided foundational knowledge of platelet interactions with cancer cells, new preclinical models are still needed that can provide a reductionist approach to systematically investigate tumor-platelet-drug crosstalk.

Microphysiological models, also termed organ-on-chip platforms, are particularly well suited for mimicking longitudinal cancer events and preclinical drug discovery as they offer a bottoms-up (simple to incrementally complex) and dissectible approach in biological system design, define tumor microenvironment and three-dimensional architecture, and enable imaging and molecular analyses with high spatiotemporal resolution. Currently, there is an unmet need for in vitro microphysiological models that can integrate the analysis of vascular and blood compartments to that of the tumor microenvironment. The studies with these models can be conducted using exclusively human-derived primary tumor and blood cells or their CRISPR gene-edited counterparts. Prior studies demonstrated the utility of organ-on-chip for faithfully modeling vascular dysfunction and platelet hyperactivity. More recently, it was demonstrated that ovarian cancer chip (termed OvCa-Chip) reproduces platelet extravasation from the blood vessel into tumors. But the prior design could not analyze the post-platelet-extravasation events that potentially can increase cancer cell proliferation, induce chemoresistance, and enhance metastasis. This was partly because OvCa-Chip lacked the technological capability of including a tumor microenvironment that can visualize and analyze invasion dynamics.

Addressing this critical deficiency here, a Tumor MicroEnvironment Organ-on-Chip (TME-Chip) was engineered, which in addition to the tumors interfacing platelet-perfused vascular endothelial tissue also incorporates an adjacent well-defined collagen hydrogel based extracellular matrix (ECM) microenvironment. This novel integration of a highly organized hydrogel architecture adjacent to the tumor cell chamber enables precise visualization of cancer cell invasion dynamics in response to the biophysical and biological effects of platelets extravasated through the endothelium into the tumor microenvironment. Using CRISPR/Cas9 knockout cancer cells, the platform revealed that platelets might promote ovarian cancer metastasis and chemoresistance through a shear-dependent interaction of their GPVI with galectin-3 expressed on the cancer cells. Finally, a novel treatment opportunity was explored to arrest ovarian cancer metastasis and increase the effects of chemotherapy via an antiplatelet drug that is relatively safe and currently undergoing clinical trials for other disease conditions. These results were further validated by performing next generation sequencing (NGS) and subsequent differential gene expression (DGE) analysis of platelet-interacted cancer cells extracted from the TME-Chip. Taken together, this work suggests that TME-Chip, integrated with gene editing and next gen RNA-seq tools, is a platform to advance the discovery of novel antiplatelet therapeutics against tumor metastasis and chemoresistance.

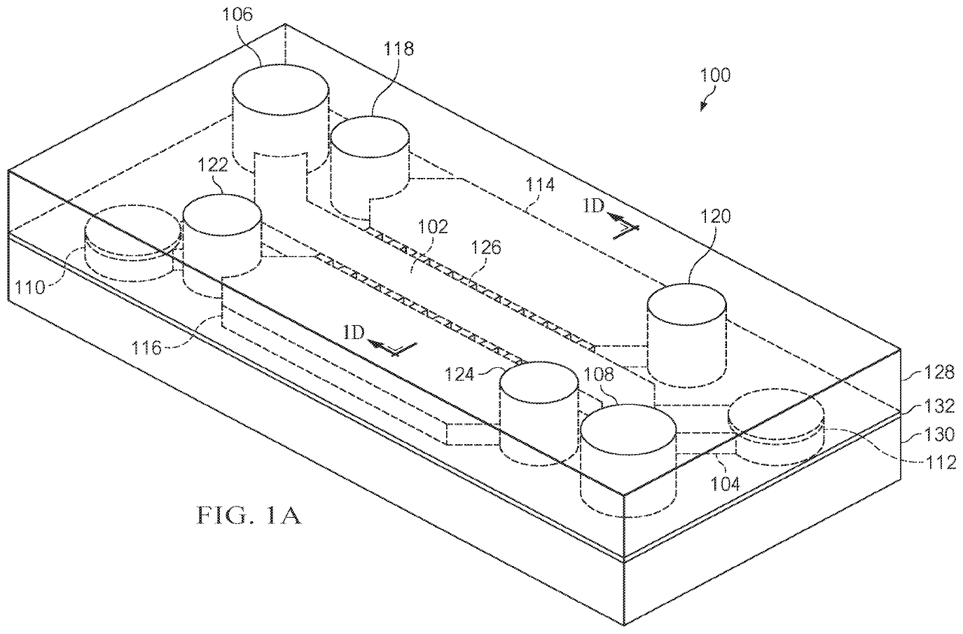

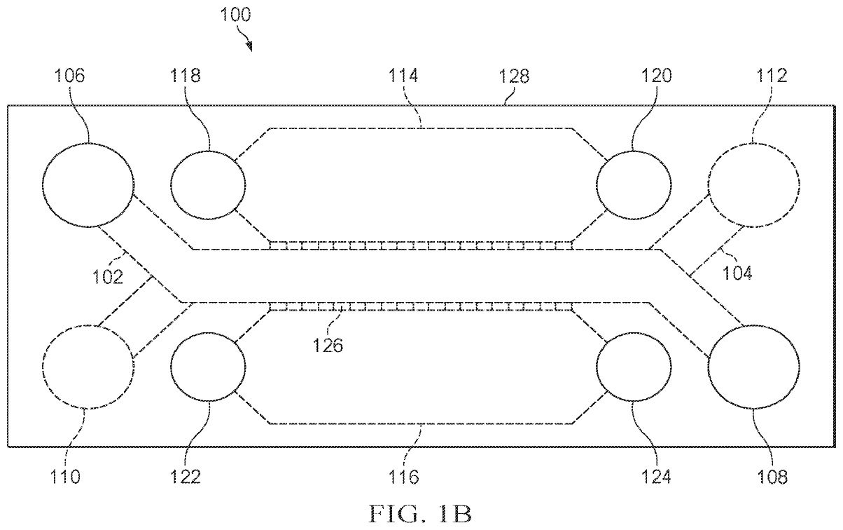

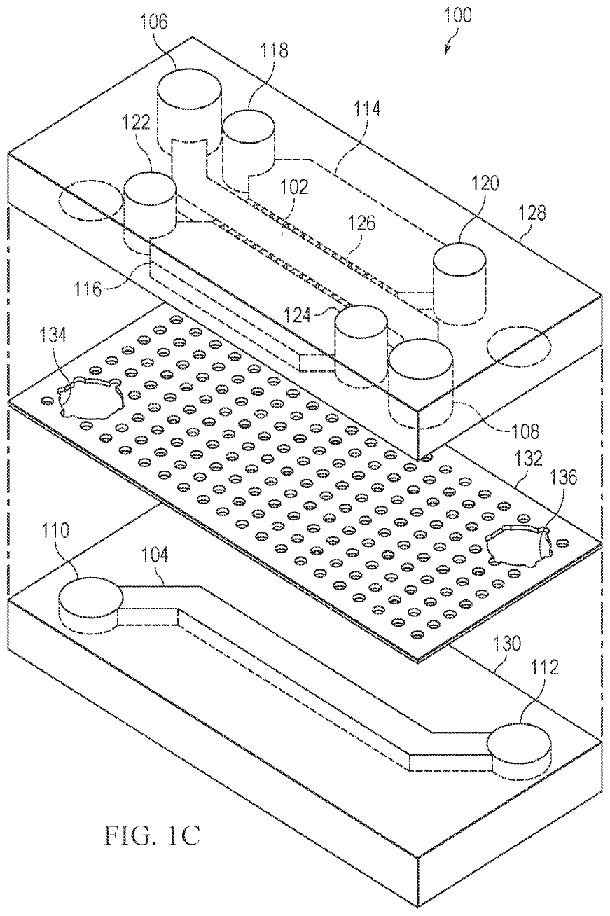

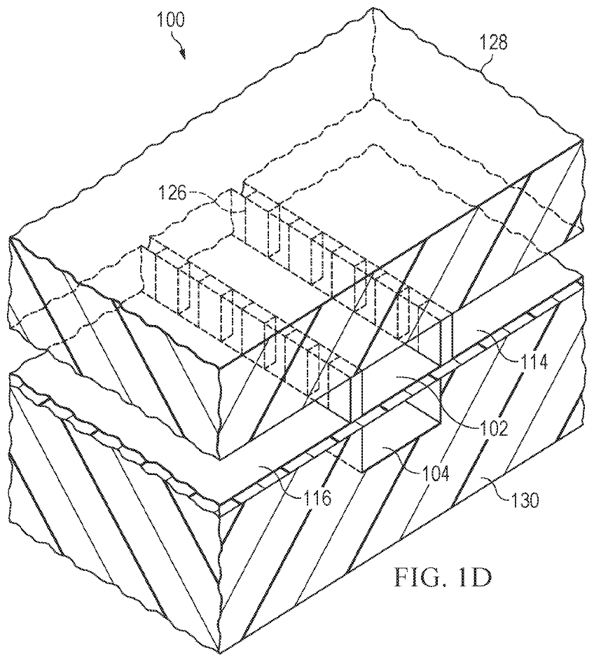

FIGS. 1A-1D illustrate a microdevice 100 in the form of a TME-Chip according to embodiments of the disclosure. FIG. 1A is a perspective view of microdevice 100, FIG. 1B is a top view of microdevice 100, FIG. 1C is an exploded assembly of microdevice 100, and FIG. 1D is a detail view of microdevice 100. Microdevice 100 includes a first chamber 102 positioned above a second chamber 104. First chamber 102 (e.g., a tumor chamber) is formed through a PDMS slab 128 and includes an inlet 106 and an outlet 108. Second chamber 104 (e.g., a vascular chamber) is formed through a PDMS slab 130 and includes an inlet 110 and an outlet 112. Micro device 100 includes two microfluidic chamber 114, 116 that are positioned on either side of first chamber 102. The two microfluidic chambers 114, 116 may be extracellular matrices (ECM) chambers. Microfluidic chamber 114 includes an inlet 118 and an outlet 120. Microfluidic chamber 116 includes an inlet 122 and an outlet 124. Microfluidic chambers 114, 116 are separated from first chamber 102 by a plurality of spaced apart micropillars 126. FIG. 1D is a detail view of chambers 102, 104, 114, 116 and micropillars 126. A membrane 132 (best seen in FIG. 1C) is positioned between PDMS slabs 128, 130 and includes cutouts 134, 136 that align with inlet 110 and outlet 112, respectively. Membrane 132 is a matrix-coated PDMS membrane that is perforated. Microdevice 100 is described in more detail in the Working Examples section.

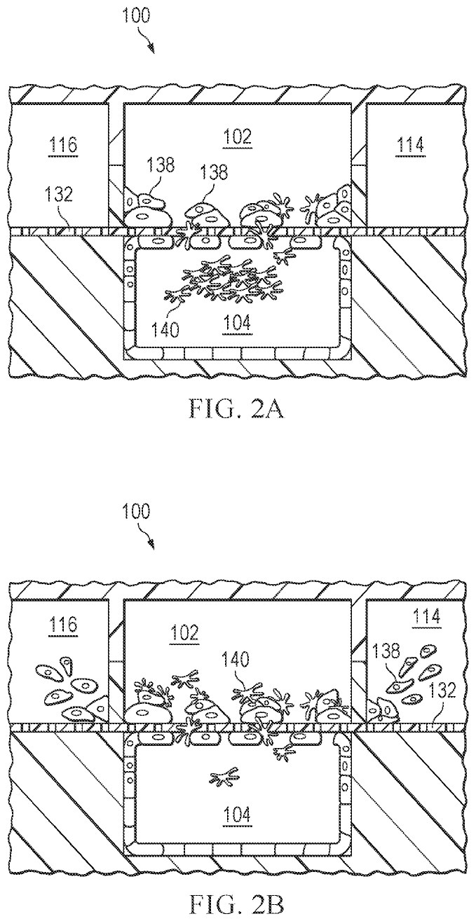

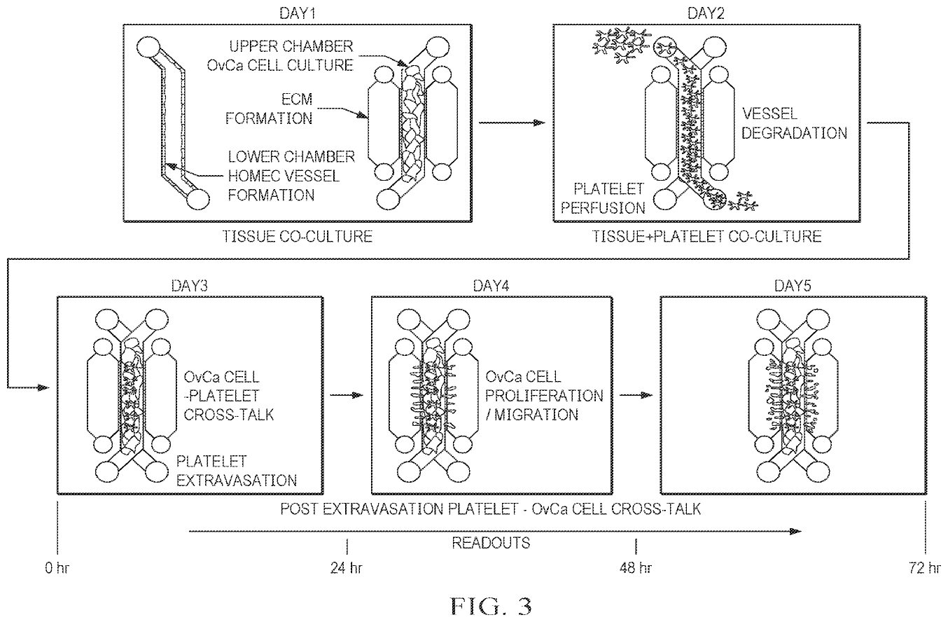

FIGS. 2A and 2B are sectioned side views of microdevice 100 illustrating platelet triggered OvCa cell migration. In FIG. 2A, cancer cells 138 have begun migrating toward microfluidic chambers 114, 116 and platelets 140 have begun extravasating into first chamber 102. In FIG. 2B, cancer cells 138 have begun migrating into microfluidic chambers 114, 116 and platelets 140 have continued migrating into first chamber 102.

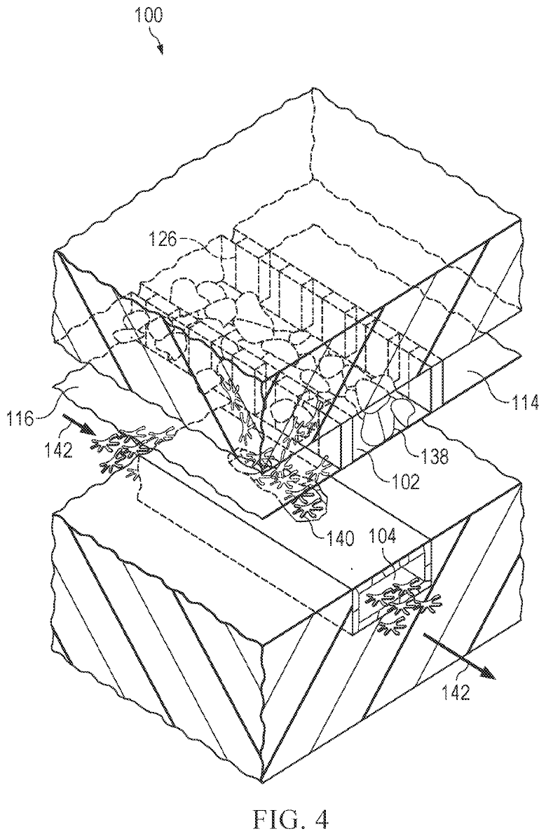

FIG. 3 is a timeline illustrating steps of TME-Chip formation, platelet extravasation into the tumors, and following consequences. FIG. 3 illustrates progression after over a five-day period. Cellular and molecular analytics were performed every 24 hrs. FIG. 4 is a schematic diagram of microdevice 100 illustrating tumor invasion dynamics. Arrows 142 indicate the direction of flow of platelets 140 through second chamber 104. In FIG. 4, extravasation of platelets 140 is occurring and cancer cells 138 are migrating past micro pillars 128 into microfluidic chambers 114, 116.

### WORKING EXAMPLES

Reference will now be made to more specific embodiments of the present disclosure and data that provides support for such embodiments. However, it should be noted that the disclosure below is for illustrative purposes only and is not intended to limit the scope of the claimed subject matter in any way.

Formation of ovarian tumor microenvironment-on-chip (OTME-Chip). Cumulative data inspired the design of an organ-chip microfluidic platform that served as a 3D organomimetic in vitro model of the tumor microenvironment, interfaced with a blood vessel and flow of platelets. Here, the primary goal was to enable the investigation of cancer cell-platelet dynamics after platelets extravasate through the endothelium into the ovarian tumors. A few elements were kept of the existing platform of an OvCa-Chip—a two-chamber organ-on-a-chip composed of cancer cells co-cultured with a 3D endothelial vessel, separated by a matrix-coated PDMS membrane (e.g., see FIGS. 1A-1D)—but to enable longitudinal studies of cancer progression and to analyze the effect of extravasated platelets on cancer cell proliferation and invasiveness, the top tumor chamber 102 of device 100 was completely reengineered by adding two adjacent ECM chambers 114, 116 separated by an array of PDMS micropillars 126 on the sides. This biosystem design strategy, now termed Ovarian Tumor Micro Environment-Chip (OTME-Chip), was inspired by a different organ-on-a-chip technology that modeled metastatic invasiveness of colorectal and breast cancer cells through artificially created ECM-mimicking materials. This design doesn't permit blood perfusion or an intimate analysis of the interactions between the cancer cells, vascular cells and blood constituents. However, in the OTME-Chip, the fusion of OvCa-Chip to this pillar-structure design provides us the first opportunity to investigate the critical platelet-cancer cell interactions under flow, while still keeping the technology relatively simple and easy to adapt. First, microfluidic chambers 114, 116 were established by injecting a pre-gel solution made of collagen type I and incubated the chip to form a semisolid hydrogel limited by micropillars 126. Next, the lower chamber 104 of microdevice 100 was seeded with human ovarian microvascular endothelial cells (HOMECs) to form an intact 3D vascular lumen. This was followed by the seeding of A2780 epithelial ovarian cancer cells in the central region of the top tumor chamber 102 that formed the interface of a blood vessel and ovarian tumor tissue with the extracellular matrix. Further, this co-culture was sustained with perfusion of freshly derived platelets from human blood inside the vascular lumen under microvascular shear for three days. Within three days, platelets increasingly extravasate into the ovarian cancer cells through the endothelium within the device.

After the platelets have extravasated into tumor chamber 102, focused was then drawn on new analyses on platelet interactions with cancer cells and consequences. The co-culture of the ovarian cancer cells was further progressed with extravasated platelets together inside OTME-Chip for another three days with media perfusion that is typical of ovarian interstitial flow (˜0.5-1 dyne cm⁻²). Cellular and molecular analytics was then performed every 24 hr (FIG. 3). Post platelet extravasation from the vessel into tumor chamber 102, invasion of cancer cells through micropillars 126 into the laterally created hydrogel ECM chambers 114, 116 in the OTME-Chip (FIG. 4) was observed. In contrast, no such invasion by cancer cells within ECM chambers 114, 116 was detected when they were kept devoid of platelets inside tumor chamber 102 (Control-Chip). Therefore, these data support the ability of the platform to model platelet-tumor effects within the tumor microenvironment.

Platelets interact with cancer cells through binding of GPVI to galectin-3 under shear. In several cancers, including ovarian cancer, the expression of galectins is upregulated and induce platelet adhesion and hyperactivity. Adhered and activated platelets, in turn, prime cancer cells for metastasis. Tumor galectin-3 specifically contains a collagen-like domain. On the other hand, the collagen receptor on platelets, GPVI, regulates platelet activation under shear. Therefore, it was examined whether OTME-Chip may reveal the contribution of GPVI-galectin-3 binding to the platelet-cancer cell interaction and its metastatic consequences under shear. The expression and activity of galectin-3 was first examined over the course of the 72 hr timeline of exposure of cancer cells to extravasated platelets and found that galectin-3 expression was conserved and did not change over time.

Next, the expression of platelet GPVI within OTME-Chip was analyzed. Since platelet GPVI is known to be shear-sensitive, it was decided to compare platelet GPVI expression under variable shear. It was found that when the platelets were perfused at a typical physiological shear (˜1 dyne cm⁻²), GPVI expression was high, but in contrast, both static condition as well as perfusion at a supraphysiological shear (˜5 dyne cm⁻²) resulted in a diminished GPVI expression. The data suggest that at low physiological shear, GPVI-galectin-3 interactions may be potent due to the high availability of GPVI.

Thereafter, to evaluate platelet GPVI binding with tumor galectin-3, immediately after exposing the platelets to shear within the OTME-Chip and extravasation, the GPVI protein was isolated and purified from the platelets and galectin-3 from the ovarian cancer cells. A rapid increase in the binding signal was detected when platelet-derived GPVI protein was perfused on an SPR Chip coated with galectin-3 protein isolated from the tumors. In contrast, the binding was absent when a GPVI free platelet lysate (immune-pulled down) was used, and significantly diminished when an inhibitory anti-GPVI monoclonal antibody was perfused along with GPVI. In summary, the analyses suggests platelet GPVI binds to tumor galectin-3.

Consequences of GPVI and galectin-3 interaction in ovarian cancer assessed with OTME-Chip. The possibility that the GPVI-galectin-3 interaction may serve as a mediator for platelet-promoted tumor metastasis in ovarian cancer was then investigated. To test this hypothesis, an ovarian cancer cell line with galectin-3 knocked out (KO-g3) was established using the CRISPR-Cas9 editing method. The parameters that govern cancer cell metastasis (ECM invasion, proliferation, cytokine profile, and transcriptional readouts) in OTME-Chips that were either composed of original ovarian cancer cells (OTME-Chip) or galectin-3 knocked down cancer cells (KO-OTME-Chip) were compared. It was found that rapid hydrogel ECM invasion by the wild-type ovarian cancer cells when co-cultured with extravasated platelets (OTME-Chip) relative to controls devoid of platelets (Control-Chip). However, it was observed that diminished ECM invasion in KO-OTME-Chips in the presence of extravasated platelets. Cancer cell proliferation increased in OTME-Chips relative to controls during 40-72 hr of extravasated platelets and tumor interaction, and proliferation was found diminished in galectin-3 KO-OTME-Chips. It was observed that this OvCa cell proliferation to be proportional to their ECM hydrogel invasion within the observation timeline.

G₂/M extension and reduced apoptosis are a hallmark of metastasis of several carcinomas. Cell cycle analysis of these OTME-Chips showed rapid alterations in cell cycle phases. A prominent G2/M phase extension and reduced apoptosis were observed in WT ovarian cancer cells due to platelets (OTME-Chip) as compared to their platelet less counterpart (Control-Chip), and this extended G2/M was found to be reduced in KO-g3 cancer cells even in the presence of platelets (KO-OTME-Chip.

The effluents obtained from the tumor chambers of OTME-Chips were examined and a time-dependent increase in the concentration of growth factors PDGF, VEGF and TGFβ and cytokines IL-8, MMP9 and IL-6 in effluents was found due to extravasated platelets that were significantly reduced in KO-TME-Chips (FIG. 3F).

It was also hypothesized that GPVI-galectin-3 interaction eventually leads to the activation of the downstream NF-kβ pathway in cancer cells. This NF-kβ signaling acts in combination with TGF-β/SMAD pathway to trigger tumor metastasis and rapid proliferation. Gene expression analysis of TME-Chips revealed a rapid upregulation in the expression of proliferation and metastasis genes in the wild-type ovarian cancer cells due to extravasated platelets compared to galectin-3 KO cancer cells and platelet-less controls. A time-dependent rapid increase in the expression of BCL2, MMP9, E-CADHERIN, AKT, NF-Kβ, SNAIL, and a moderate increase in VIMENTIN, C-MYC, CASP, CYCLIN1, SMAD, BAX, CDK4, LYNK1, and MMP2 like hallmark cell proliferation and metastasis regulator genes are observed in cancer cells over the co-culture time due to platelets. This data strengthens the characterization of GPVI-galectin-3 interaction as an initiating factor behind the transcriptional signaling that is necessary for the proliferation and metastasis of ovarian cancer.

OTME-Chip predicts that pharmaceutical inhibition of GPVI arrests metastasis and supports chemotherapy. The potential consequences of pharmaceutical targeting of GPVI on platelets in cancer were then investigated. There are currently no FDA-approved anti-GPVI drugs in the market. However, Revacept is an inhibitor that has been successfully evaluated against collagen-mediated platelet adhesion in phase I clinical trials of atherosclerosis and stroke. Revacept inhibited the platelet GPVI and tumor galectin-3 interaction in a colon carcinoma cell line. This drug is a fusion protein consisting of the Fc part of human IgG, a short hinge region, a specific linker sequence, and the extracellular part of human platelet GPVI with a molecular mass of 150 kDa. It was hypothesized that the specific inhibition of platelet GPVI with antiplatelet drug Revacept would arrest the tumor's metastatic potential within the in vitro OTME-Chip. Subsequent to platelet perfusion in OTME-Chip and extravasation of platelets into the ovarian cancer cells, the tumors were exposed to Revacept at its clinically relevant concentration (40 μg/mL). Thereafter, these chips were analyzed for three days (FIG. 7). It was found that Revacept-treated OTME-Chips (Rₓ-OTME-Chip) exhibited significantly reduced GPVI-galectin-3 binding kinetics. Revacept significantly reduced ECM invasion and proliferation rate of the cancer cells, suggesting that GPVI inhibition was also preventing the consequence of GPVI-galectin-3 interaction. To confirm that the results are not dependent on A2780 cancer cells alone, a moderately invasive ovarian cancer cell line OVCAR3 was also tested and again observed an increased cancer cell invasion due to platelets but inhibited by Revacept.

Cell cycle analysis revealed a reduced G2/M phase in cancer cells from Rₓ-OTME-Chips compared to untreated controls. Further, Revacept reduced the growth factors (PDGF, VEGF, and TGFβ) and cytokines (IL-8, MMP9, and IL-6). The transcriptional analysis of cancer cells obtained from the chips showed that the platelet-induced upregulation of metastasis and cell proliferation genes declined rapidly over time in Revacept-treated Rₓ-OTME-Chips.

Furthermore, since platelets have been shown to promote tumor chemoresistance, the effect of GPVI-galectin-3 interaction and blocking this interaction by Revacept on chemoresistance in ovarian cancer cells was examined. The OTME-Chips were treated with either no drug (OTME-Chip) or anticancer drug cisplatin alone near its clinically relevant concentration (6 μg/mL) (Cₓ-OTME-Chip), or cisplatin compounded with Revacept (CₓRₓ-OTME-Chip). It was found that cisplatin alone had a modest effect, whereas the cisplatin-Revacept combination had a significant effect in reducing tumor invasiveness, cell proliferation, G2/M phase, and concentration of growth factors and cytokines. Finally, transcriptional analysis of cancer cells obtained from the chips showed the platelet triggered upregulation of metastasis and cell proliferation genes altered overtime modestly when cisplatin alone is included within OTME-Chips (Cₓ-OTME-Chip); however, Revacept significantly attenuated this overexpression (CₓRₓ-OTME-Chip). Altogether, the OTME-Chip's capability to assess anticancer drugs systematically provides us these preclinical datasets, which suggest that GPVI inhibition could be a potential strategy against ovarian cancer metastasis and chemoresistance.

Validation of cancer cell-platelet-drug crosstalk within OTME-Chip with next generation RNA-sequencing. It was recently shown that a collaboration of organ-chip technology with RNA-seq analysis is a powerful tool to validate preclinical studies and derive unbiased clinical predictions. Therefore, to solidify insights into the platelet-tumor signaling nexus and the therapeutic effect of Revacept, transcriptomic profiling was performed of different chip-derived tumors through RNA sequencing. Through RNA sequencing and differential gene analysis, expression of 12452 genes in the OTME-Chip relative to Control-Chip was detected. Similarly, expression of 5566, 6909 and 3821 genes in the Cx-OTME-Chip, CxRx-OTME-Chip and KO-OTME-Chip respectively relative to Control-Chip was detected.

The reduction in differentially expressed genes in the different drug treated and knockout conditions suggest that the drug treatments were attenuating the transcriptomic expression and transformed the physiological behavior of the chips close to untreated controls. Among the different chip conditions, there were 15 groups of genes that were either unique to each condition or were common between chip conditions for the different permutations. Comparing genes common to two chip conditions at a time, it was observed that the OTME-Chip and CxRx-OTME-Chip conditions had the highest number of common genes (˜5500). In addition to the NFκB and TGFβ/Smad pathways that have been elucidated before, and in this work, the RNA-seq analysis allowed us to investigate other signaling pathways that were upregulated in the different chip conditions via KEGG pathway clustering. When compared to the platelet free tumors (Control-Chip) it was observed that in addition to the already known NFκB and TGFβ/Smad pathways, EMT and metastasis regulatory signaling pathways like Hippo (KEGG:04390), MAPK (KEGG:04010), mTOR (KEGG:04150), Notch (KEGG:04330), PI3-Akt (KEGG:04151) and Wnt (KEGG:04310) pathways were upregulated. This result suggests that targeting these other pathways could further open new avenues towards the development of anticancer therapeutics. To further visualize the efficacy of different treatments, volcano plots were generated and genes belonging to the aforementioned signaling pathways were highlighted. As expected, both the chemotherapy drug cisplatin and the antiplatelet Revacept reduced the expression of these genes. This downregulation in expression was also confirmed by generating heatmaps showing holistic differences in expression profiles of the different chip conditions (FIG. 6F). Interestingly, OTME-Chip had a complementary expression profile compared to the Control-Chip. Consequently, it was observed that cisplatin therapy downregulated the expression of these pathways and that a combination therapy of cisplatin and Revacept further reduced their expression. This downregulation in the CxRx-OTME-Chip was close to the Galectin-3 knockout cancer cells available from KO-OTME-Chip where the GPVI-Galectin-3 interaction was disturbed via knockout. This result unbiasedly validates that GPVI-Galectin-3 interaction is pivotal in platelet mediated tumor promotion and establishes the therapeutic efficacy of antiplatelet drug Revacept against the progression of cancer with this preclinical platform.

Although several other groups have pioneered the creation of bioengineered models of tumors alone, tumor-vessel constructs, and tumor-ECM constructs, the primary focus of these studies did not include examining platelet-tumor interactions and consequences. Notably, platelets are one of the first blood cells to interact with cancer cells, and it is increasingly getting appreciated clinically that thrombocytosis is associated with a poor prognosis in several cancers. Specifically, in ovarian cancer, animal models convincingly support that platelets extravasate into the ovarian tumor microenvironment (OTME) and increase proliferation and epithelial-mesenchymal transition (EMT) in ovarian cancer cells. In the current study, prior expertise in leveraging organ-chip technology was built upon to integrate blood-cell function into modeling processes in cancer. Here, a novel OTME-Chip was created that demonstrates a biomimicry of the consequences of the platelet-cancer cell interaction in ovarian cancer hard to achieve via conventional in vivo or in vitro models. The addition of hydrogel compartments adjacent to the cancer cell-vessel interface within the chip permitted time-lapse visualization of perfused platelets within the vessel, their extravasation through the endothelium, and consequential cancer cell invasiveness that was not previously obtained through ectopic in vivo experiments. Furthermore, isolating cancer cells from the chip during different time points of platelet perfusion have allowed us to identify the platelet-mediated increase in tumor proliferation and specific alterations in cancer cell cycle phases. Although this study is limited to ovarian cancer, this platform can be expanded to other cancer models, and include more complex formulations of matrices and interstitial microenvironment.

It was demonstrated that GPVI is a critical mediator in the adhesion of extravasated platelets to cancer cells. The counter receptor for GPVI is galectin-3 that is expressed on cancer cells. It was shown that platelet GPVI expression is shear-dependent, and therefore, its interaction with the tumors could be dependent upon the hemodynamic state. Replacing wild-type cancer cells with galectin-3 knockout cells in OTME-Chip identified the key regulatory role of GPVI-galectin-3 interaction in metastasis. The in vitro results are supported by several clinical observations and animal studies. For example, overexpression of galectin-3 increased tumor burden in A2780 ovarian cancer xenografted mice. Increased expression of galectin-3 has also been detected in advanced stages in ovarian cancer patients. Increased levels of soluble GPVI have also been reported in plasma samples. GPVI blockade inhibited lung, colon, and breast cancer metastasis in mouse models. Studies in colorectal and breast cancer have described an increase in growth factors and cytokines in the TME after platelet extravasation into the tumors, and this has been predicted due to GPVI galectin-3 interaction. In ovarian cancer, a similar elevation of growth factors and cytokines in TME is observed due to extravasated platelets. Also, the extravasated platelets have been identified to cause proliferation of tumors via TGF-β/SMAD pathway, but the regulation of GPVI galectin-3 interaction on activation of NF-kβ signaling hasn't been explored. Analysis of effluents from tumor microenvironment collected from the chips during the hours of platelet-tumor interaction by transcriptomics and multiplexing methods detected alterations in gene expression and protein profile. This identified the upregulation of a series of metastasis triggering growth factors and cytokines due to GPVI-galectin-3 interaction.

Antiplatelet therapy has been tested in several clinical trials of cancer in the past. For example, acetylsalicylic acid (ASA) or aspirin has been suggested to reduce platelet aggregation on tumors and inhibit metastasis. In an in vitro study, aspirin and P2Y₁₂ inhibition attenuated platelet-induced ovarian cancer cell invasion. However, these drugs have a major drawback that they can increase bleeding risk in patients. Therefore, antiplatelet therapeutics that may be anti-metastatic but do not alter hemostasis is an important unmet need. Platelet GPVI is an excellent target since it is known to be antithrombotic, but does not cause bleeding. With the OTME-Chip, preclinical evidence that Revacept inhibited platelet-cancer cell interactions and its consequences was provided, and therefore, is a potential drug to be advanced into clinical trials of ovarian cancer. Finally, it was shown that the GPVI-galectin-3 interaction induces chemoresistance in cancer cells, and Revacept can inhibit this effect. The inhibitory effect of Revacept treatment on cancer cell invasion independently as well as in combination with cisplatin was also verified in detail through the RNA sequencing analysis. Therefore, this study has a translational value in the context that it motivates Revacept to be tested in a clinical trial against ovarian and other cancers.

Although in this study, the focus was on cancer metastasis triggered by the extravasated platelets, it is possible that blood immune cells like tumor-associated macrophages, myeloid-derived suppressor cells, and regulatory T cells can also support tumor metastasis by forming a cumulative signaling along with platelets that remodel the tumor microenvironment. These immune cells could be included in this OTME-Chip study to identify their influence on tumors and metastasis. Pericytes while forming the vascular tissue inside the chip was not include, and pericytes can potentially exert influence on platelet extravasation. Additionally, whole blood in the vascular chamber were not perfused because it is very difficult to maintain blood fluidity in vitro for a prolonged period. Finally, unlike some other tumor models where cancer cells are fully embedded within the matrix, cancer cells in the model were seeded over the matrix to prevent non-specific interactions between platelets and cancer cells, and to enable robust visual and molecular analytics. This relative simplicity and a provision to do analytics is a strength of the OTME-Chip which is also not radically different from its prior versions and consistent with prior models. Overall, this preliminary device design is a suitable proof-of-concept that highlights out OTME-Chip as a powerful tool in the investigation of blood cell influence on cancer and metastasis. An interesting future opportunity this platform offers is to also include the interaction of platelets with circulating tumor cells that may possibly disseminate from the solid tumor into the vascular chamber of this chip.

In conclusion, it has been demonstrated that complex interaction between the tumors and blood platelets, and the resulting metastatic process could be modeled in a bioengineered tumor microenvironment with vascular components on a chip. It was uncovered that this platform could be used in the identification of complex tumor-blood cell interactions, metastatic signaling, and the evaluation of drugs with an integrated genomic and proteomic approach. In addition, the scope of application of desired anticancer or antiplatelet drugs individually or in combination also makes the device a potential platform to perform drug evaluation studies against platelet triggered metastasis and chemoresistance of cancer. Opportunity to include ECM in the chip also added additional capability to investigate the tissue invasiveness and invasion behavior of tumors due to platelets under different conditions. It is expected that this OTME-Chip technology could further be advanced towards personalized cancer medicine by integrating iPSCs derived from malignant tumors and other patient-derived blood or immune cells, where the interaction signaling underlying metastasis can be dissected.

In summary, this human OTME-Chip model may offer a potential platform for studying the interaction between blood cells and cancer cells, acquiring functional readouts, and pre-clinical data of different anti-metastasis drugs before initiating large animal studies and human clinical trials.

### Working Examples Materials and Methods

Cell lines. Human Ovarian Microvascular Endothelial Cells (HOMECs; Sciencell Research Laboratories) were cultured in Endothelial Cell Medium (ECM, Cat. #1001) and ovarian carcinoma A2780 cells (SigmaAldrich, Cat #93112519) were cultured in RPMI 1640 medium. Each were supplemented with 100 U/mL Penicillin and 100 μg/mL Streptomycin. Tumor galectin-3 knockout A2780 cells were developed using CRISPR/Cas9 methods and selected on Puromycin (1 mM). Single guide ribonucleic acids (gRNA) targeting the exon 3 of galectin-3 were designed for the knock-out study. The designed guides were screened in silico for off-target using COSMID activity webtool and the gRNA (CATGATGCGTTATCTGGGTC) with the least number of off-targets was selected for the 5 knock-out experiments. The guide was delivered as a ribonucleoprotein complex (RNP) with Cas9 to the A2780 cell line using an optimized nucleofection protocol.

Human platelets. Platelets were isolated from fresh human donor blood samples following previously mentioned protocol using acid citrate dextrose (ACD) buffer and HEPES-Tyrode's buffer to preserve their cytoskeletal activity. Platelets were tagged using Phycoerythrin (PE) conjugated CD-41 monoclonal antibody (ThermoFisher Scientific, CAS-VIPL3; dilution1:200) before perfusion into the device (˜200×10³ platelets μL⁻¹). Blood samples were obtained via phlebotomy according to the policies of the US Office of Human Research Protections (OHRP) and approved by the Texas A&M University Institutional Review Board (IRB) (ID: IRB2016-0762D).

Drugs. Cisplatin (Fisher Scientific, USA) and Revacept (Advancecor GamBH, Germany) were dissolved in DMSO and PBS/4% Mannitol/1% Sucrose respectively to prepare master stocks (1 mg/mL). These were further diluted in RMPI-1640 media to obtain respective working concentrations of cisplatin (6 μg mL⁻¹) and Revacept (40 μg mL⁻¹). They were perfused into the chip tumor chamber individually or in combination in presence or absence of extravasated platelets.

Design und fabrication of the microfluidic OTME-Chip. OTME-Chips were adapted from previously reported OvCa-Chips, where the top portion of the device was changed from a single microchannel (1 mm length×1 mm wide×100 μm height), to three parallel microchannels. The three microchannels were separated by hexagonal micropillars instead of a continuous PDMS wall, allowing for the formation of hydrogel-fluid barriers that supported cell invasion. Each micropillar had dimensions 250 μm×100 μm×100 μm (length×width×height) and were equally spaced leaving 100 μm between each micropillar. Drawings of the device were made using SolidWorks 2019, from which photomasks were printed. Master molds were made using traditional soft lithography procedures. PDMS slabs and porous membrane were casted and cured, assembled, and processed according to previously developed protocols.

Pre-gelled hydrogel solutions were prepared on ice by neutralizing 3.57 mg mL⁻¹ rat-tail collagen I (Corning) with 1 N NaOH before diluting to 2.5 mg mL⁻¹ with cell culture medium. 5 μL of the pre-gelled hydrogel solution were injected into each ECM channel and the device was stored in incubator at 37° C. for 1 hr for gel solidification. After gelation, hydrogel channels were hydrated by injecting a mixture of rat tail collagen type-I (100 μg mL⁻¹; BD Biosciences) and fibronectin (30 μg mL⁻¹; BD Biosciences) inside the tumor and vessel chambers of chip and incubated at 37° C. for at least 2 hours. This step also prepared cellular compartments for cell seeding. Before loading cells into their respective chambers, a wash with 1×PBS was performed to remove unbound residual collagen. HOMECs and A2780 OvCa cells were harvested and centrifuged, their supernatant removed, and pellets resuspended in respective fresh culture media before loading into devices. HOMEC cell suspension (5×10⁵ cells mL⁻¹) was injected inside the vessel chamber and incubated at 37° C. by following previously protocols to obtain a confluent microvessel of HOMECs. After the microvessel was formed, the collagen coated upper tumor chamber of the chip was seeded with cancer cell suspension (5×10⁵ cells mL⁻¹) and cultured for 24 hr to obtain a confluent monolayer. Platelets were perfused inside the vascular chamber using a syringe pump. The shear stress reported is at the wall that was calculated from the Navier-Stokes's equation applicable for simple rectangular channels, as follows:

$${τ}_{w} = \frac{6 η Q}{{h}^{2} w} \qquad Eq . 1$$

where τ_{w} is wall shear stress, η is dynamic viscosity of fluid, Q is the flowrate, and h and w are microfluidic channel's height and width, respectively. Post-platelet extravasation, the cancer cells inside the tumor chamber were cultured under a continuous flow of RPMI-1640 media for another 3 days (˜1 dynecm⁻²). Tumor chamber media effluent was collected from the devices at 24, 48, and 72 hr. Cancer cells and platelets were isolated from the tumor chamber using Accutase™ treatment and the CD41 tagged platelets were subsequently pulled down of from the cancer cell-platelet mixture by using MojoSort™ Human anti-CD41 magnetic Nanobeads (BioLegend, USA) on a magnetic separator provided by the manufacturer. The magnetically separated platelet and tumors fractions were washed and kept for further downstream assays.

Immunostaining and FACS. Platelets and cancer cells isolated from the device were washed by centrifugation (300 RCF for 5 min) and resuspended in phosphate-buffered saline (1×PBS), followed by blocking with 2% BSA for 10 min and fixing with 2% Paraformaldehyde for 15 min at room temperature. Fixed cells were permeabilized using 0.1% TritonX 100 for 5 mins before immunostaining. Platelets were immunostained for GPVI surface marker by using anti-human GPVI (Sigma Aldrich, Cat #ABS446) primary antibody followed by incubation with appropriate fluorescent secondary antibody as per experimental need. Fixed and permeabilized cancer cells were stained for galectin-3 protein using anti-human galectin-3 primary antibody (ThermoFisher Scientific, Cat #A3A12) and compatible fluorescent tagged secondary antibody before flow cytometry experiments. All flow cytometric analyses were performed using BD Accuri C6 flow cytometer (BD Biosciences, USA) and data was processed using CellQuest Pro software.

Cell invasion. Cancer cells were live-stained with PKH67 cytotracker-green (Sigma Aldrich, USA) before seeding into the device. Post-extravasation, the ovarian cancer cells were incubated at 37° C. with continuous perfusion of media in tumor chamber for 3 days. Invasion dynamics of live stained cancer cells (green) into the Chip ECM chamber hydrogel was visualized using fluorescence microscopy (Zeiss Axio Observer; LD Plan Neofluar 10×, NA 0.4). Snapshots were taken at every 12 hr interval with an exposure time of 200 ms. Images were analyzed and processed using software ZEN 2.3 lite (ZEISS). Measurement of cancer cell invasion through the hydrogel was performed using cell counter plugins in ImageJ. The ECM invasion of cancer cells was calculated as the ratio of area occupied by invaded cancer cells in ECM chamber vs. the total area of ECM chamber.

Cancer cell proliferation assay. Isolated cancer cells were immunostained with monoclonal anti-Ki67 antibody-Alexa Fluor488 conjugate (Abcam). The fluorescence intensity was then measured with a plate reader using excitation/emission wavelengths of 488/519 nm. Platelet free cancer cells obtained from other chips during similar time points were kept as control. Proliferation rate was measured as a percentage ratio of fluorescent intensities of experimental and control cells.

Cell cycle analysis. Cancer cells isolated via Accutase™ treatment from the chip were fixed in 70% ethanol at 20° C. for 6 hr and permeabilized. Cells were then incubated with 100 μg mL⁻¹ Propidium Iodide (PI) (Sigma-Aldrich), and 50 μg mL⁻¹ ribonuclease A in the dark for 30 min at 37° C. Flow cytometry was performed on a BD Accuri C6 flow cytometer (BD Biosciences, USA) and cell cycle analysis was done using CellQuest Pro software.

Growth factors and cytokines. Growth factors and cytokines present in OTME-Chip tumor chamber media effluents were measured using multiplex (xMAP) magnetic bead-based technology provided by Millipore. Media effluents collected from tumor chamber during different time points were analyzed for cytokines and growth factors using MILLIPLEX® MAP Human cytokine/chemokine magnetic bead panel kit (Millipore, CAS-HCYTOMAG-60K) by following previously established protocols. Assays were run in a pre-calibrated Luminex MAPGPIX reader. The mean fluorescent intensity (MFI) of the magnetic beads correlating with the concentration (pg/mL) of respective cytokines and growth factors was calculated using xPONENT 4.2 software and data analysis was done with Milliplex Analyst 5.1 software (Millipore). The concentration of cytokines obtained in each sample was normalized with respect to controls and the assays were run in triplicate. Cytokine data for each group were normalized to corresponding cell number respectively.

Quantitative real-time PCR. Cancer cell total RNA was isolated using the Arcturus PicoPure RNA Isolation Kit specifically (Thermofisher Scientific, CAS-KIT0214) designed to recover high-quality RNA consistently from fewer cells. Cells isolated from the Chip were washed in prescribed in buffer (0.9 mL of 1×PBS/10% BSA; 0.1 mL of 0.5 M EDTA) and total cellular RNA was isolated by using the manufacturer's protocol. Isolated RNA was treated with DNAase and its quality was checked to be standard 260/280>1.5 with spectrophotometry before 0.5 μg of RNA was processed further for the preparation of cDNA using ProtoScript® First Strand cDNA Synthesis kit (NEB). The cDNA prepared was further used for the subsequent quantitative qRT-PCR reactions using SyBR green master mix (Applied Biosystems™ SYBR™ Green select PCR Master Mix) and gene specific primers. PCR Reactions for respective genes were performed in qRT-PCR system (Thermo Fisher Applied BioSystems Quantstudio 3) keeping GAPDH as an internal control. Gene expression was analyzed by quantifying the relative fold changes in mRNA levels of individual genes using comparative Cycle Threshold (CT)/ACT method.

Western blot. Galectin-3 knockdown in cancer cells were verified by western blot assay. In brief, cell lysates were prepared from equal amount of wild type and knockout A2780 cells and protein extraction was performed using spin columns. Equal amount of protein (20 μg) from harvested cells were loaded onto 10-15% w/v polyacrylamide gels and separated by SDS-PAGE, followed by transfer to polyvinylidene fluoride (PVDF) membrane. Post-transfer, membrane was incubated with anti-galectin-3 antibody (Thermofisher, CAS-A3A12, 1:10,000) and anti-β actin antibody (Thermofisher, CAS-PA1-183, 1:10,000 dilution), followed by an incubation with HRP-conjugated anti-rabbit immunoglobulin G (Abcam, CAS-ab6721 1:5,000 dilution) secondary antibody. The protein bands were then visualized with chemiluminescence (BioRad, USA), and densitometry quantification was done by using ImageJ analysis software.

Surface plasmon resonance. Physical interaction between platelet surface GPVI and tumor galectin-3 was determined via level free analysis using surface plasmon resonance method (Nicoyalife, USA). Galectin-3 protein was pulled down from cancer cell and platelet lysates using Biotinylated anti-galectin-3 monoclonal antibody and anti-GP-VI monoclonal antibody, respectively. The proteins were diluted in HBS-EP-EDTA running buffer (0.01 M HEPES, 0.15M NaCl, 3 mM EDTA, pH 7.4). Streptavidin coated chips purchased from Nicoyalife were used to immobilize biotinylated galectin-3 by injecting the protein into the device for 5 min using a flow rate of 20 μl/min, thus immobilizing biotinylated galectin-3 on the streptavidin coated chip. Successful immobilization was confirmed by observation of an increase ˜50 RU in the sensorgram signal. Purified GPVI (100 μL) was injected at a flow rate of 20 μl min⁻¹ over the galectin-3 immobilized chip and the resonance unit change in the sensorgram signal was recorded. For inhibition studies, 40 μg mL⁻¹ Revacept was added with purified GPVI and injected into the device. The data obtained was analyzed by curve fitting of the Langmuir binding isotherm with the software.

RNA sequencing and analysis. RNA from the chips were isolated according to the method described earlier. Isolated mRNA samples were then analyzed using the NextSeq 500 platform (Illumina) and sample preparation was performed using TruSeqRNA sample preparation with paired-end read length of 2×75 bases (Molecular Genomics Workspace, Texas A&M University, College Station, TX). Then HISAT2 was used to splice align the reads to latest ENSEMBL-release-102 human genome/transcriptome (GRCh38.p13). To generate raw counts from alignment files (SAM), the Bioconductor package was used SUBREAD. Differentially expressed genes were then evaluated for all groups using DESeq2 package in R®. The cutoff to determine significant genes in all groups were FDR adjusted p-value (q-value)<0.05. Finally, KEGG pathway analysis was performed using the online database DAVID (Database for Annotation Visualization and Integrated Discovery, v6.8) and visualized using Cytoscape and ClueGO.

Statistical Analysis. All results are presented as means±SEM, unless otherwise noted. Data were analyzed with using one-way analysis of variance (ANOVA) followed by Dunnett's multiple comparisons test using GraphPad Prism (GraphPad Software, San Diego, CA, USA). p<0.05 was considered as statistically significant.

In brief, disclosed herein illustrates the engineering an Ovarian Tumor MicroEnvironment Organ-on-Chip (OTME-Chip), which in addition to the tumors interfacing platelet-perfused vascular endothelial tissue, also incorporates an adjacent well-defined collagen hydrogel based extracellular matrix (ECM) microenvironment. This novel integration of a highly organized hydrogel architecture adjacent to the tumor cell chamber enables precise visualization of cancer cell invasion dynamics in response to the biophysical and biological effects of platelets extravasated through the endothelium into the tumor microenvironment. Using CRISPR/Cas9 knockout cancer cells, this platform revealed that platelets might promote ovarian cancer metastasis and chemoresistance through a shear-dependent interaction of their GPVI with galectin-3 expressed on the cancer cells. Finally, a novel treatment opportunity was explored to arrest ovarian cancer metastasis and increase the effects of chemotherapy via an antiplatelet drug that is relatively safe and currently undergoing clinical trials for other disease conditions. These results were further validated by performing next generation sequencing (NGS) and subsequent differential gene expression (DGE) analysis of platelet-interacted cancer cells extracted from the OTME-Chip. Taken together, this work suggests that OTME-Chip, integrated with gene editing and next gen RNA-seq tools, is a platform to advance the discovery of novel antiplatelet therapeutics against tumor metastasis and chemoresistance. This inspired the design of an organ-chip microfluidic platform that served as a 3D organomimetic in vitro model of the tumor microenvironment, interfaced with a blood vessel and flow of platelets. Here, the primary goal was to enable the investigation of cancer cell-platelet dynamics after platelets extravasate through the endothelium into the ovarian tumors. A few elements of the existing platform were kept from a OvCa-Chip-a two-chamber organ-on-a-chip consisting of cancer cells co-cultured with a 3D endothelial vessel, separated by a matrix-coated PDMS membrane—but to enable longitudinal studies of cancer progression and to analyze the effect of extravasated platelets on cancer cell proliferation and invasiveness, the top tumor chamber of the device was completely reengineered by adding two adjacent extracellular matrices (ECM) chambers separated by an array of PDMS micropillars on the sides. This biosystem design strategy, now termed Ovarian Tumor Micro Environment-Chip (OTME-Chip), was inspired by a different organ-on-a-chip technology that modeled metastatic invasiveness of colorectal and breast cancer cells through artificially created ECM-mimicking materials. This design doesn't permit blood perfusion or an intimate analysis of the interactions between the cancer cells, vascular cells and blood constituents. However, in the OTME-Chip, the fusion of OvCa-Chip to this pillar-structure design provides the first opportunity to investigate the critical platelet-cancer cell interactions under flow, while still keeping the technology relatively simple and easy to adapt.

First, the ECM compartments were established by injecting a pre-gel solution made of collagen type I and incubated the chip to form a semisolid hydrogel limited by micropillars. Next, the lower chamber of the device was seeded with human ovarian microvascular endothelial cells (HOMECs) to form an intact 3D vascular lumen. This was followed by the seeding of A2780 epithelial ovarian cancer cells in the central region of the top tumor chamber that formed the interface of a blood vessel and ovarian tumor tissue with the extracellular matrix. Further, this co-culture was sustained with perfusion of freshly derived platelets from human blood inside the vascular lumen under microvascular shear for three days. Within three days, platelets increasingly extravasate into the ovarian cancer cells through the endothelium within the device.

After the platelets have extravasated into the tumor chamber, the focused shifted to new analyses on platelet interactions with cancer cells and consequences. The co-culture of the ovarian cancer cells were further progressed with extravasated platelets together inside OTME-Chip for another three days with media perfusion that is typical of ovarian interstitial flow. Cellular and molecular analytics was then performed every 24 hr. Post platelet extravasation from the vessel into the tumor compartment, invasion of cancer cells through the pillars into the laterally created hydrogel ECM in the OTME-Chip was observed. In contrast, no such invasion by cancer cells within ECM was detected when they were kept devoid of platelets inside the tumor chamber (Control-Chip). Therefore, these data support the ability of the platform to model platelet-tumor effects within the tumor microenvironment.

As such, disclosed herein is a medical device containing four compartmentalized microfluidic channels. Two channels are situated vertically and separated by a porous membrane so that living cells may migrate between the two channels. The lower channel contains a blood vessel-mimicking endothleium and the top channel contains cancer cells. The top channel is flanked by two microchannels that contain a biologically derived hydrogel scaffold. Unlike the upper and lower channels, the lateral channels are separated by a plurality of micropillars at a set distance that facilitates the formation of a solid-liquid interface, through which two-dimensional monolayers of tumor cells can assume a three-dimensional morphology after undergoing epithelial-to-mesenchmyal transition (EMT).

TME-Chips were advancements of prior organ-on-a-chip art, where the top portion of the device was changed from a single microchannel (1 mm length×1 mm wide×100 μm height), to three parallel microchannels. The three microchannels were separated by hexagonal micropillars instead of a continuous PDMS wall, allowing for the formation of hydrogel-fluid barriers that supported cell invasion. Each micropillar had dimensions 250 μm×100 μm×100 μm (length×width×height) and were equally spaced leaving 100 μm between each micropillar. Drawings of the device were made using SolidWorks 2019, from which photomasks were printed. Master molds were made using traditional soft lithography procedures. PDMS slabs and porous membrane were casted and cured, assembled, and processed according to previously developed protocols.

Pre-gelled hydrogel solutions were prepared on ice by neutralizing 3.57 mg mL⁻¹ rat-tail collagen I (Corning) with 1 N NaOH before diluting to 2.5 mg mL⁻¹ with cell culture medium. 5 μL of the pre-gelled hydrogel solution were injected into each ECM channel and the device was stored in incubator at 37° C. for 1 hr for gel solidification. After gelation, hydrogel channels were hydrated by injecting a mixture of rat tail collagen type-I (100 μg mL⁻¹; BD Biosciences) and fibronectin (30 μg mL⁻¹; BD Biosciences) inside the tumor and vessel chambers of chip and incubated at 37° C. for at least 2 hr. This step also prepared cellular compartments for cell seeding. Before loading cells into their respective chambers, a wash with 1×PBS was performed to remove unbound residual collagen. HOMECs and A2780 OvCa cells were harvested and centrifuged, their supernatant removed, and pellets resuspended in respective fresh culture media before loading into devices. HOMEC cell suspension (5×10⁵ cells mL⁻¹) was injected inside the vessel chamber and incubated at 37° C. by following previously protocols to obtain a confluent microvessel of HOMECs. After the microvessel was formed, the collagen coated upper tumor chamber of the chip was seeded with cancer cell suspension (5×10⁵ cells mL⁻¹) and cultured for 24 hr to obtain a confluent monolayer. Platelets were perfused inside the vascular chamber using a syringe pump

Although others have pioneered the creation of bioengineered models of tumors alone, tumor-vessel constructs, and tumor-ECM constructs, the primary focus of these studies did not include examining platelet-tumor interactions and consequences. Notably, platelets are one of the first blood cells to interact with cancer cells, and it is increasingly getting appreciated clinically that thrombocytosis is associated with a poor prognosis in several cancers. Specifically, in ovarian cancer, animal models convincingly support that platelets extravasate into the ovarian tumor microenvironment (TME) and increase proliferation and epithelial-mesenchymal transition (EMT) in ovarian cancer cells. In the current study, the devices disclosed herein were built upon prior expertise in leveraging organ-chip technology to integrate blood-cell function into modeling processes in cancer. Here, a novel TME-Chip was created that demonstrates a biomimicry of the consequences of the platelet-cancer cell interaction in ovarian cancer hard to achieve via conventional in vivo or in vitro models. The addition of hydrogel compartments adjacent to the cancer cell-vessel interface within the chip permitted time-lapse visualization of perfused platelets within the vessel, their extravasation through the endothelium, and consequential cancer cell invasiveness that was not previously obtained through ectopic in vivo experiments. Furthermore, isolating cancer cells from the chip during different time points of platelet perfusion have allowed for the identification of the platelet-mediated increase in tumor proliferation and specific alterations in cancer cell cycle phases.

Organ-on-chip methods have revolutionized the drug discovery process, and biomedical startups, pharmaceutical industry and the Food and Drug Administration are using these platforms as a way to design new drugs, assess drug and chemical toxicities, radiation exposure and countermeasures and facilitate clinical trials bypassing or complementing small and/or large animal studies. The devices disclosed herein show a new method designed to advance cancer discovery and therapeutics. The devices herein add new capabilities to those previously available, for example, the provision of a new microfluidic compartment lateral to the direction of flow and cell culture to observe cancer cell growth, invasion and metastasis

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

What is claimed is:

1. A medical device comprising:
a first compartmentalized microfluidic channel flanked by two microfluidic chambers;
a second compartmentalized microfluidic channel, wherein the first and second compartmentalized microfluidic channels are situated vertically relative to one another; and
a porous membrane situated between the first and second compartmentalized microfluidic channels.

2. The medical device of claim 1, wherein the first and second compartmentalized microfluidic channels are in fluid communication such that cells can migrate from the first compartmentalized microfluidic channel to the second compartmentalized microfluidic channel through the porous membrane.

3. The medical device of claim 1, wherein the first compartmentalized microfluidic channel comprises cancer cells.

4. The medical device of claim 1, wherein the second compartmentalized microfluidic channel comprises blood vessel-mimicking endothelium.

5. The medical device of claim 1, wherein the two microfluidic chambers comprise a biologically derived hydrogel scaffold.

6. The medical device of claim 5, wherein the biologically derived hydrogel scaffold permit for at least one of time-lapse visualization of perfused platelets and blood immune cells within a vessel, their extravasation through an endothelium, or consequential cancer cell invasiveness.

7. The medical device of claim 1, further comprising lateral channels separated by a plurality of micropillars.

8. The medical device of claim 7, wherein the plurality of micropillars are at a set distance to facilitate formation of a solid-liquid interface.

9. The medical device of claim 8, wherein the solid-liquid interface facilitates in two-dimensional monolayers of tumor cells assuming a three-dimensional morphology.

10. The medical device of claim 9, wherein the three-dimensional morphology is assumed after undergoing epithelial-to-mesenchmyal transition (EMT).

11. The medical device of claim 1, wherein the medical device identifies an antiplatelet therapeutic, and wherein the antiplatelet therapeutic comprises Revacept.

12. An organ-on-chip comprising:
a top portion comprising three parallel microchannels, wherein the three parallel microchannels are separated by a plurality of micropillars;
a bottom portion comprising a fourth microchannel; and
a membrane between the top portion and the bottom portion.

13. The organ-on-chip of claim 12, wherein the plurality of micropillars comprises hexagonal micropillars.

14. The organ-on-chip of claim 12, wherein each micropillar has dimensions of 250 μm×100 μm×100 μm (length×width×height).

15. The organ-on-chip of claim 12, wherein each micropillar of the plurality of micropillars is equally spaced apart.

16. The organ-on-chip of claim 12, wherein the top portion comprises cancer cells.

17. The organ-on-chip of claim 12, wherein the bottom portion comprises blood vessel-mimicking endothelium.

18. The organ-on-chip of claim 12, wherein the plurality of micropillars comprises a biologically derived hydrogel scaffold.

19. The organ-on-chip of claim 18, wherein the biologically derived hydrogel scaffold permits for at least one of time-lapse visualization of perfused platelets and blood immune cells within a vessel, their extravasation through an endothelium, or consequential cancer cell invasiveness.

20. The organ-on-chip of claim 12, wherein the organ-on-chip identifies an antiplatelet therapeutic, and wherein the antiplatelet therapeutic comprises Revacept.
